(19) United States
(12) Patent Application Publication
(10) Pub. No.: US 2018/0042543 A1
(43) Pub. Date: Feb. 15, 2018

(54) APPLICATION FOR SCREENING VESTIBULAR FUNCTIONS WITH COTS COMPONENTS

(71) Applicant: Charles River Analytics, Inc., Cambridge, MA (US)

(72) Inventors: Michael P. Jenkins, Boston, MA (US); Arthur Wollocko, Fairfax, VT (US); Scott Irvin, Cambridge, MA (US); Henry Adams Roth, Cambridge, MA (US)

(21) Appl. No.: 15/673,898

(22) Filed: Aug. 10, 2017

Related U.S. Application Data
(60) Provisional application No. 62/373,083, filed on Aug. 10, 2016.

(51) Int. Cl. A61B 5/00; A61B 5/11

(52) U.S. Cl. CPC A61B 5/4023; A61B 5/11; G06F 19/322

(57) ABSTRACT

Systems and methods are disclosed that record quantifiable data for physical exams that assess neurological function. A system includes four main components. First, it employs a flexible and customizable procedure administration and documentation system developed and deployed on a mobile platform to aid in the identification, administration, configuration, and instruction of a suite of procedures for assessing different aspects of vestibular health. Second, it leverages commercial off-the-shelf (COTS) hardware with integrated sensor technology to allow non-vestibular experts to conduct assessment procedures by imposing constraints that ensure accurate and safe administration of VF assessment procedures. Next, it utilizes a gaming engine to both capture patient responses and to enable the accurate visual presentation of required stimuli for each of its assessments. Lastly, it leverages database storage and retrieval to visualize and aggregate data from multiple assessments and over many trials.

### CROSS-REFERENCE TO RELATED APPLICATION

This application is based upon and claims priority to U.S. Provisional Patent Application No. 62/373,083, entitled “Application for Screening Vestibular Functions with COTS Components,” filed Aug. 10, 2016, attorney docket number 75426-47; the entire contents of this prior application are incorporated herein by reference.

### STATEMENT REGARDING FEDERALLY SPONSORED RESEARCH

This invention was made with government support under government contract number W81XWH-15-C-0041 awarded by the United States Army Medical Research Acquisition Activity (USAMRAA); and, further under government contract number W81XWH-16-C-0070 awarded by the United States Army Medical Research Acquisition Activity (USAMRAA). The government has certain rights in the invention.

### BACKGROUND

### Technical Field

This disclosure relates to systems that record quantifiable data for physical exams that assess neurological function.

### Description of Related Art

In prior art systems, a patient's performance in physical exams was typically assessed via observation, such as a physician, and therefore measures of performance were heavily dependent on the experience and knowledge of the observer. The subjectivity of exam performance-assessment meant that determining the improvement/decline of a patient's neurological condition across different tests and different test-administers was essentially impossible.

For example, vestibular function tests (“VFTs”) are commonly used to determine the health of the vestibular portion of the inner ear, since that portion of the inner ear allows a person to sense the orientation of his or her body with respect to gravity. The vestibular system also allows and is used by a person to adjust the body's orientation with respect to self-generated movements, as well as forces that are exerted upon the person's body from the outside world. The vestibular system performs these essential tasks by engaging a number of reflex pathways that are responsible for making compensatory movements and adjustments in body position.

Some VFTs are used to determine if a subject's dizziness, vertigo, or balance problem is caused by a brain disorder or trauma. These tests have typically been conducted in controlled clinical environments by trained otolaryngologists or audiologists using costly, specialized medical screening equipment. This has limited the ability of first responders to carry out any sort of robust screening or triage for vestibular dysfunction at the point of injury, often resulting in a failure to recognize the subtle symptoms of vestibular injuries that can be present directly following a head impact or barotrauma.

Thus, there is a heretofore unmet need for providing responders access to systems that effectively guide them through the appropriate vestibular screening techniques support them in the diagnosis of vestibular dysfunction at the point of injury, and assist with the administration and future assessment of these procedures.

### SUMMARY

The systems and methods of the present disclosure solve this problem by providing quantifiable measures of exam performance, enabling repeatable, consistent assessment of performance for a number of neurological function tests aimed at assessing vestibular function.

An aspect of the present disclosure is directed to a software framework—a software program or set of coordinated and cooperating programs—tailored for smartphone devices that enables rapid development, integration, and deployment of various stimulus-response (SR) based trials used to assess an individual's health.

The present disclosure provides systems that record quantifiable data for physical exams that assess neurological function. Such systems include four main components. First, a flexible and customizable procedure administration and documentation system is employed which is developed and deployed on a mobile platform to aid in the identification, administration, configuration, and instruction of a suite of procedures for assessing different aspects of vestibular health. Second, commercial off-the-shelf (COTS) hardware with integrated sensors, e.g., inertial measurement units (“IMUs”), are used to allow non-vestibular experts to conduct assessment procedures, with the sensors imposing constraints that ensure accurate and safe administration of VF assessment procedures. Next, the system utilizes a gaming engine (software program running on a suitable processor) to both capture patient responses and to enable the accurate visual presentation of required stimuli for each of its assessments. Lastly, the system employs a database for storage and retrieval to visualize and aggregate data from multiple assessments and over many trials.

An exemplary embodiment presents a system for deploying stimulus-response (SR) based health assessment methods for assessing the health of a subject. The system includes a flexible and customizable procedure administration and documentation user interface architecture operative, e.g., via software applications resident on a smart device, to present a plurality of health assessment procedures to an evaluator. The system further includes a virtual reality environment configured to enable the accurate audiovisual presentation of stimulus for different health assessments to trigger responses from a subject. The system includes a plurality of positional sensors operative to acquire data of the subject's stimulus-responses. The system further includes a computer-readable non-transitory storage medium, including computer-readable instructions; and a processor connected to the memory and operative to evaluate the subject's stimulus-responses, wherein the processor, in response to reading the computer-readable instructions, is operative to: evaluate the subject's stimulus-responses, and present the evaluation to the evaluator. The system can further utilize a database, such as implemented on a backend server operating in conjunction with the smart device.

A further exemplary embodiment presents computer-readable non-transitory storage media including computer-readable instructions for implementing the instructions via use a suitable processor accessing the instructions resident in the computer-readable storage media.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1A depicts a diagram of an example of the functional engineering architecture of the ADVISOR framework system.

FIG. 1B diagrammatically shows implementation 100B of the ADVISOR system on a smart device as used by a responder to assess the health of subject wearing a fieldable screening kit.

FIGS. 1C-1F together depict an example end-to-end workflow through an embodiment of the ADVISOR suite for an embodiment of the present disclosure.

FIG. 2 depicts an example of file storage based on passed filenames for an embodiment of the present disclosure.

FIG. 3 depicts an example of flexible documentation framework for providing in-depth instructions and setup requirements for a particular procedure for an embodiment of the present disclosure.

FIG. 4 depicts an example of a flexible documentation framework architecture, detailing the database specifications file for each procedure, an example of the ADVISOR assessment display parser that ingests information and maps tagged content to UI elements, and an example of a resulting ADVISOR generated user interface for an embodiment of the present disclosure.

FIG. 5 depicts an example of ADVISOR ray casting and collision library that allows for the tracking of patient head movement by casting rays into the virtual environment originating from the focal eye points (represented by the camera) for an embodiment of the present disclosure.

FIG. 6 depicts an example control interface that can be used to manipulate a target's trajectory and different trials within an assessment for an embodiment of the present disclosure.

FIG. 7 depicts example statistics for a single trial generated by the Review Performance capability for an embodiment of the present disclosure.

FIG. 8 depicts an example of wireframe components used for embodiments of the present disclosure.

FIG. 9 depicts an example of upper spine extension and flexion in a wireframe model according to the present disclosure.

FIG. 10 depicts an example of rotational measurement of wire frame components according to the present disclosure.

FIG. 11 depicts an example of upper arm vertical abduction and adduction for a wireframe model according to the present disclosure.

FIG. 12 depicts an example of upper arm extension and flexion for a wireframe model for a wireframe model according to the present disclosure.

FIG. 13 depicts an example of upper arm horizontal adduction and abduction for a wireframe model according to the present disclosure.

FIG. 14 depicts lower arm extension and flexion for a wireframe model according to the present disclosure.

FIG. 15 depicts lower arm horizontal adduction and abduction for a wireframe model according to the present disclosure.

FIG. 16 depicts an example of hand flexion and extension for a wireframe model according to the present disclosure.

FIG. 17 depicts an example of wrist horizontal abduction and adduction for a wire frame model according to the present disclosure.

FIG. 18 depicts an example of upper leg flexion and extension for a wire frame model according to the present disclosure.

FIG. 19 depicts an example of upper leg adduction abduction for a wire frame model according to the present disclosure.

FIG. 20 depicts an example of lower leg flexion and extension for a wire frame model according to the present disclosure.

FIG. 21 depicts an example of foot plantar flexion and dorsiflexion for a wire frame model according to the present disclosure.

FIG. 22 depicts recorded data for left and right foot motion on the Y axis (forward and back) for an embodiment of the present disclosure.

FIG. 23 depicts recorded data for left and right foot motion on the X axis (left and right) for an embodiment of the present disclosure.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

As indicated above, an aspect of the present disclosure is directed to a software framework tailored for smartphone devices that enables rapid development, integration, and deployment of various stimulus-response (SR) based trials used to assess an individual's health. Exemplary embodiments of the present disclosure include a flexible and customizable procedure administration and documentation system is employed which is developed and deployed on a mobile platform—such as a smart device including but not limited to a tablet or a smartphone—to aid in the identification, administration, configuration, and instruction of a suite of procedures for assessing different aspects of vestibular health. Commercial-off-the-shelf (COTS) hardware with integrated sensors, e.g., inertial measurement units (“IMUs”), are used to allow non-vestibular experts to conduct assessment procedures, with the sensors imposing constraints that ensure accurate and safe administration of VF assessment procedures. Next, a gaming engine (software program running on a suitable processor) is employed to both capture patient responses and to enable the accurate visual presentation of required stimuli for each of its assessments. Lastly, the system employs a database (e.g., resident on a backend server) for storage and retrieval to visualize and aggregate data from multiple assessments and over many trials.

Responders need access to systems that effectively guide them through the appropriate vestibular screening techniques support them in the diagnosis of vestibular dysfunction at the point of injury, and assist with the administration and future assessment of these procedures. Soldiers suffering a traumatic brain injury (TBI) or barotrauma need accurate, timely, in-theater assessment of symptoms to inform appropriate return-to-duty (RTD) decisions. Often, this initial assessment and diagnosis must be conducted by first-level responders (e.g., Medics, Corpsmen, etc.) who attempt to assess vestibular symptoms and are often present directly following a concussive event; however, these symptoms are often missed, not adequately evaluated, or misdiagnosed due to a lack of familiarity with the subtleties of impaired vestibular function. To support this need, systems and related methods of the present disclosure combine inexpensive commercial off-the-shelf hardware components with a flexible and customizable procedure administration and documentation framework and VR presentation and data presentation tools to create a robust vestibular function assessment package; including a system and method for the Assessment and Diagnosis of Vestibular Indicators of Soldiers' Operational Readiness, or “ADVISOR.” Thus, exemplary embodiments (instantiations) of the present disclosure are collectively referred to herein as “ADVISOR.” The ADVISOR package includes two full-fledged Android applications (i.e., the main Android application and Unity based VR application), and numerous “Shared Components”, the details of which are all outlined below. Of course, while ADVISOR is presented in the context of the Android operating system and platforms, other embodiments of the present disclosure can be utilized with other operating systems and platforms, e.g., iOS used on an Apple device, etc.

Examples of the ADVISOR system combine an integrated hardware platform (centered around a head-mounted display (HMD)) with automated assessment capabilities to provide very low-cost, portable screening capabilities tailored for in-theater use. ADVISOR supports:

(i) Multiple Stimulus Presentation which allows for the presentation and manipulation of the different stimuli required for each of the fifteen included vestibular assessments (e.g., Subjective Visual Vertical (SVV), Dynamic Acuity (DVAT), Vestibular Sensory Organization (VEST-SOT));

(ii) Response-Capture Modalities that support the aforementioned battery of fifteen clinically validated and novel assessment methods. The capture capabilities employed by ADVISOR utilize state-of-the-art sensing capabilities to objectively collect data on movement, position, rotation, and input timing, allowing for expert level assessments by novice personnel; and

(iii) Intuitive Test Administration and Output Interpretation Interfaces that both decrease the effort required by the medical responder to select and conduct appropriate assessments, and increase the consistency and accuracy of RTD decisions

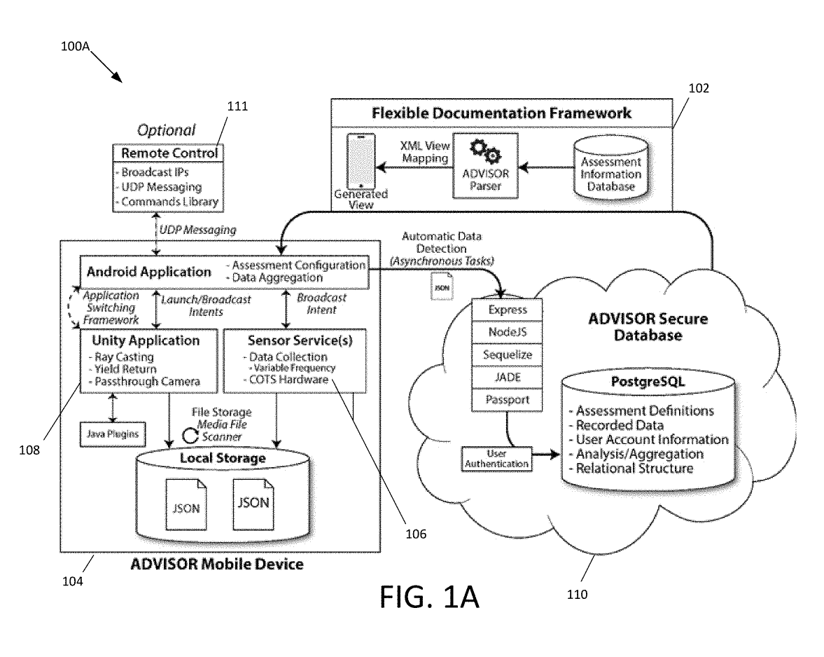

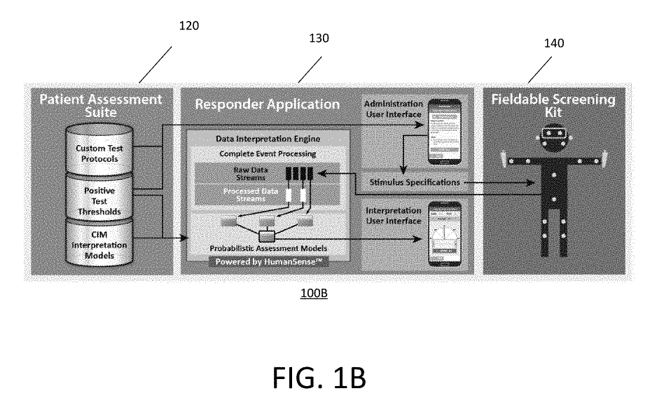

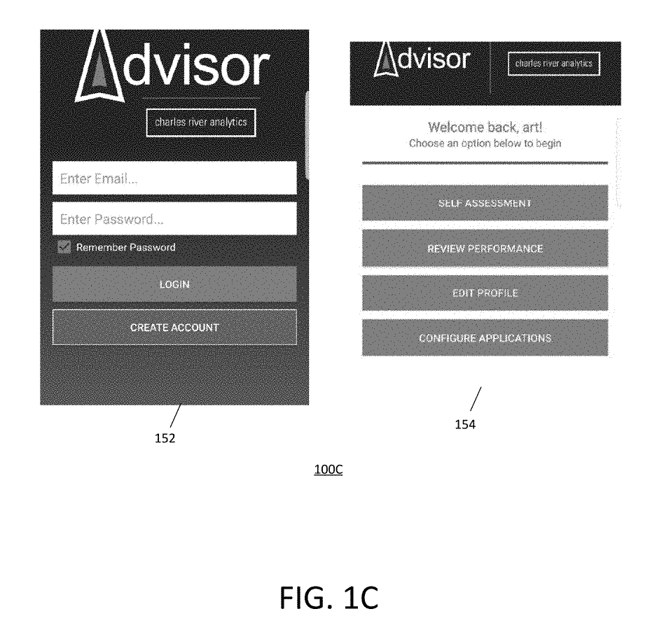

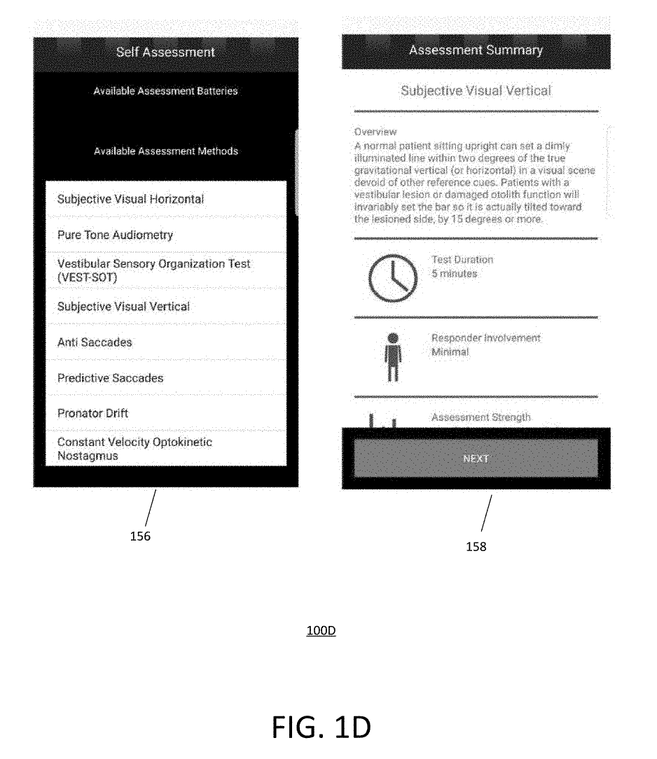

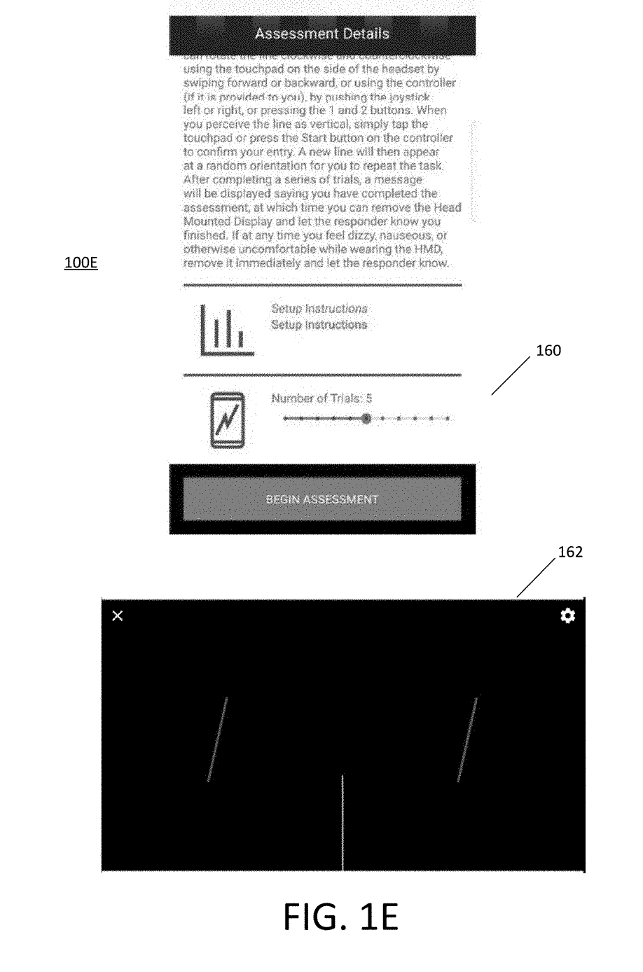

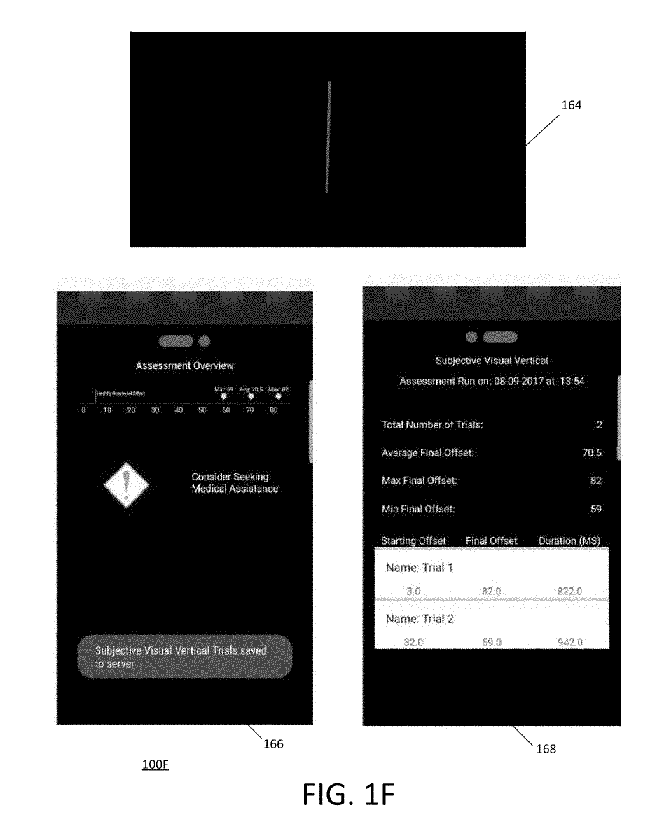

FIG. 1A depicts a diagram of an example of the ADVISOR system 100A. FIG. 1B diagrammatically shows implementation 100B of the ADVISOR system on a smart device as used by a responder to assess the health of subject wearing a fieldable screening kit. FIGS. 1C-1F together depict an example end-to-end workflow through the ADVISOR suite.

As shown in FIG. 1A, exemplary embodiments of ADVISOR's framework encompass four main components. First, a flexible and customizable procedure administration and documentation system 102 is deployed on a mobile platform 104 to aid in the identification, administration, configuration, and instruction of a suite of procedures for assessing different aspects of vestibular health. Second, commercial off-the-shelf (COTS) hardware 106 with integrated sensor technology is used, allowing non-vestibular experts to conduct assessment procedures, by imposing constraints that ensure accurate and safe administration of VF assessment procedures. Next, a gaming engine 108, such as, e.g., the Unity3D gaming engine, is utilized for the system to both capture patient responses and to enable the accurate visual presentation of required stimuli for each of its assessments. Lastly, a database 110 is utilized for storage and retrieval to visualize and aggregate data from multiple assessments and over many trials. To enable each of these components, several shared components were developed to assist in the creation of a seamless application suite. These are shared across the two applications that make up the ADVISOR suite, one being a standard Android application developed for Android SDK 23, and the other being a Virtual Reality enabled Unity3D application. The two applications are detailed below. FIG. 1B diagrammatically shows implementation 100B of the ADVISOR system on a smart device as used by a responder to assess the health of subject wearing a fieldable screening kit. As shown in FIG. 1B, a patient assessment suite 120 is linked to a responder application 130, which is linked to a fieldable screening kit 130, which is to be worn by a subject for assessment. The fieldable screening kit 130 can include a wireless stereoscopic head-mounted display (HMD), with integrated video oculography. The kit can include wide-ranging, noise-cancelling headphones. The kit 130 can also include insertional motion sensors (IMS), which can include accelerometers, gyroscopes, magnetometers, or the like. The sensors can collect data about the motion of the subject's limbs, torso, and head. The kit can include one or more inertial motion units (IMU) for receiving and processing data from the IMS. The kit can include one or more electromyography (EMG) sensors. The kit 130 can include a wireless transceiver, e.g., a Bluetooth wireless transceiver, for wireless data transmission. The kit 130 can also include a wireless controller, e.g., a Bluetooth wireless controller. A representative workflow can be visualized in FIGS. 1C-1F, with images 152-160 representing the procedure administration and documentation framework, while image 168 represents the data visualization and aggregation from data contained within a database.

### Shared Components

To achieve the functionality desired, the ADVISOR system depends on a number of components developed under this effort, collectively referred to as “Shared Components”. These reusable components allow for several required features to be implemented within the system, including access to the underlying mobile device operating system, application switching, the creation of unique user interface elements and actions, and file storage. These are detailed below:

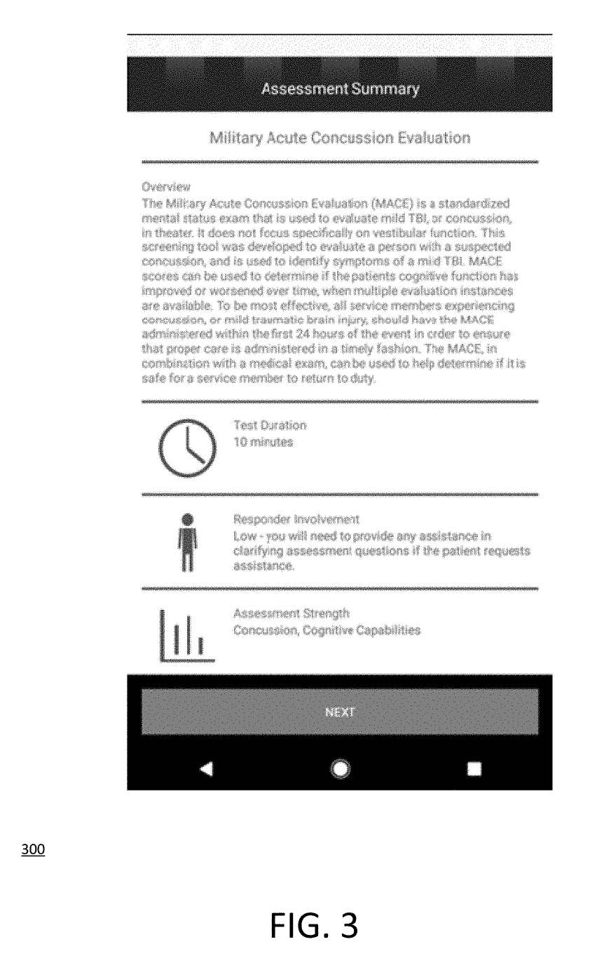

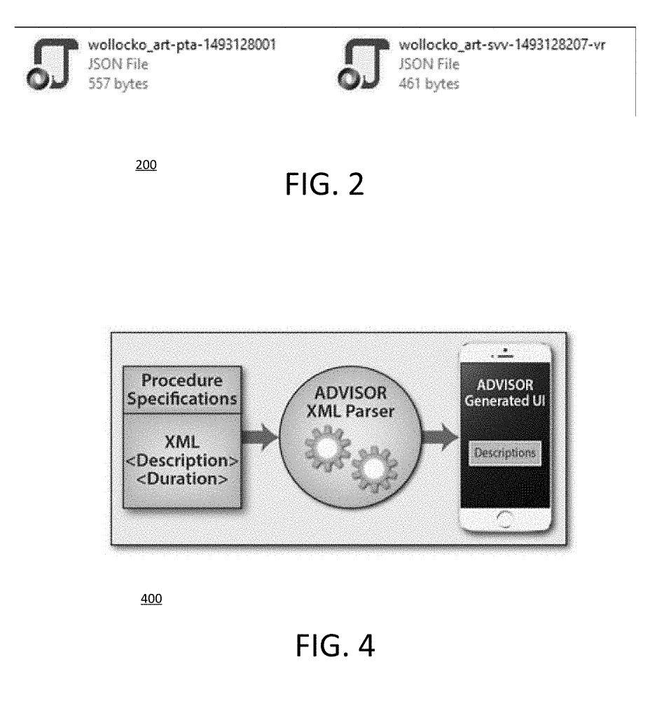

File Storage: The ADVISOR system is heavily dependent on the storage of data collected from the various COTS sensors throughout the administration of vestibular assessments. Therefore, it was important that the software be able to access the underlying file system on the mobile device, and effectively save/retrieve information. However, since ADVISOR was created to assist in medical assessment and diagnosis, it was also imperative that its design allowed for both computers and humans to ingest the stored information, affording medical responders access to the raw data associated with each assessment. Therefore, a common structured data format, JavaScript Object Notation, or JSON, was used; of course other data formats may also or in the alternative be used. Numerous open source JSON serialization libraries exist and can be leveraged to convert standard data objects like those created to hold data for each assessment into this structured format. Data captured includes primitive (string/numerical) details like the name of the procedure, duration, patient name, and then various specific data points collected that are tailored to each procedure (e.g., perceived direction of letter during DVAT examination, or distance for recorded double vision on the Test of Convergence). Then, each of these individual trials is captured in aggregated into a “Results” object, which itself is then converted to JSON. FIG. 2 depicts an example of file storage 200 based on passed filenames for an embodiment of the present disclosure. Each assessment has a unique filename generated that details some information on the assessment and the subject ID taking the assessment, and is used to generate the eventual JSON file containing all the trials for the assessment, as shown in FIG. 2. Each sensor capturing data for a particular assessment will save into its own data file, as each captures different dimensions of data. Regardless of how many sensors are involved and saving data for a particular assessment, they will all utilize the same base filename passed from the Android application, and add a suffix detailing where the data comes from (for example adding -vr for data coming from the VR HMD).

This file can either be looked at in its raw form by responders, or is displayed within the application's Results scene, which will be detailed below. Additionally, each time the file is saved by either of the two ADVISOR applications that make up the ADVISOR suite, the file is saved in an accessible location, using the underlying Android file system's predetermined ExternalStorageDirectory, ensuring each application within the suite has access to the same information throughout the entire workflow. Permissions are set on each application appropriately within the Android Manifest files, so they are able to both access the files and ensure proper data synchronization. Lastly, when saving files to the Android file system, the file storage device needs to be refreshed and rescanned, which is a common practice after doing operations like saving pictures or videos that need to be immediately accessed following their capture. If this process is not followed, the file would not be visible on the device until it was restarted. To accomplish this, every time a storage operation occurs, Android's MEDIA_FILE_SCANNER intent is used, passing in the filename of the saved data.

Application Switching: Unity3D is foremost designed as a gaming engine for the creation of PC based games. While selecting this as the design environment afforded the capabilities to design intuitive display and interactions required for each assessment, it did present several challenges. Mainly, deployment to an Android device did not grant access to the underlying features of the Android Operating System, which was a desire for the system's implementation. Due to this, the Unity application lacked the capability to register itself with, and manipulate the Android application stack, allowing for easy switching between applications. Therefore, to support this capability, an Android plugin was created as a .jar library, and included and referenced within the Unity C# code. The Application Switching Plugin was developed within Android Studio, and allows the C# code to pass an Android bundle name which represents the application you wish to launch, to this library. This bundle name is then used to launch the corresponding application (e.g., com.microsoft.skype to launch Skype). The plugin code will suspend the current Android Intent (the ADVISOR Responder Application), and place it into the background as a suspended application, storing it on the Android Application stack so it can be returned to easily with the pressing of the “Back” button on the mobile device, or through the utilization of the device's multi-tasking capabilities. Then, the plugin launches the desired application through the utilization of Intents and the provided package name. This results in a seamless switch between applications, with the ADVISOR application remaining in a suspended mode, allowing users to return to their previous location within the application.

VR Configuration Using Android Intents: While seamless application switching enables a VR/non-VR interaction with the suite, it was important to allow for dynamic configuration and communication between the two applications—As Android, and particularly Unity-based Android applications, do not share data easily. For instance, the VR application that was utilized contains implementations of all the assessments in the utilized suite, but does not know which assessment should be run unless it is configured by and directed by the Android application. In addition to guiding what assessment should be run, the Android application can dictate various configuration variables, as well as passing the filename that should be used to store data to ensure both applications are operating on the same data storage location. Therefore, Android's ability to carry information can be relied upon, and when a new launch intent is generated to bring the application to the forefront using the Application Switching shared component, all information on the assessment, its configuration specified within the Android application by the patient, and the filename are all passed to the VR application, allowing it to function properly.

Ingestion of Data from Android Intents: Each time the VR application is launched, it is defaulted to launch into a Launcher Scene, which will display the ADVISOR logo and a text-based information display to the patient. This text will ask them to launch the primary Android application if something was configured incorrectly, and the VR application will be closed. However, under normal circumstances, the VR application will only be launched from the ADVISOR Android application, which will pass data along with the launch intent. The Launcher Scene of the VR application therefore makes it its first priority to search for this data and ingest it, and then parse and route the VR application appropriately. This ingestion considers all possible data key-value pairs that can be passed from the Android application, and utilizes Unity3D's PlayerPrefs classes to store data throughout the entire upcoming session. After storing data in the PlayerPrefs, the VR application routes to the appropriate scene based on the assessment name, whose scripts are then loaded and handle the ingestion of the PlayerPrefs data appropriately, setting variables for that assessment based on those passed in. The last piece of data that is used is the filename that is passed from the Android application, which ensures that the VR application stores the file in a specific location such that can be detected by the Android application once it is re-loaded.

Automatic Data Detection and Server Data Persistence: Once the VR assessment is completed, the VR application automatically closes, and due to manipulation of the Android stack through the utilized Application Switching plugin, the ADVISOR Android application is refocused. On refocus, the application launches a thread to check for the presence of new data at the filename that was passed to the VR application. If data is present, additional Android asynchronous tasks are spawned to ingest that data, and store it to the ADVISOR secure database. Routes on the server will parse the file and the data within, correctly determining the location for file storage on the server and returning a signal to the Android application that the data has been persisted. Once persisted, the patient is notified and normal use of the application can continue.

ADVISOR Secure Database: The ADVISOR server utilizes the common server Javascript library NodeJS, additionally leveraging other libraries such as sequelize, underscore, and express. These combine to form the server routes and data parsing and storage mechanisms, which feed data into the secure PostGreSQL database. All authentication is handled through the passport library, and required for each transaction on the server.

### Flexible Documentation Framework for Procedure Administration

ADVISOR is driven primarily by its flexible and customizable procedure administration and documentation framework, a graphical user interface (UI) used for selecting and administering the various procedures included within the suite. FIG. 3 depicts an example 300 of flexible documentation framework for providing in-depth instructions and setup requirements for a particular procedure as displayed on a UI, for an embodiment of the present disclosure. This framework contains various elements, including the information identified as necessary to present to the responder, information on each examination, and all the interaction elements specific to the selected procedure's configuration. This presentation is completely database driven, with the ADVISOR database containing definitions for each of the various fields populated for each assessment. This allows the flexibility to change and alter instructions and specifications without having to re-deploy additional application assets.

Different assessment procedures are used to evaluate different dimensions of VF (e.g., evaluating utricular versus saccular function). The mapping of a specific assessment procedure to the dimension(s) of VF it can assess needs to be made explicit to allow responders to select an appropriately robust battery of procedures for evaluating a patient, based on the context of the injury, patient, and evaluation environment. However, as a goal of the ADVISOR system is to enable non-vestibular experts to select and administer procedures, the application provides all of the information necessary to select an appropriate combination of procedures to ensure all critical dimensions of VF are screened. Additionally, the context of each patient evaluation will differ, so ADVISOR supports customization of the assessment workflow for different assessment configurations (e.g., different equipment requirements, constraints on examinations, lengths of examinations, variables collected during each procedure). To meet this requirement, the system employs a highly flexible and customizable procedure administration and documentation framework that allows for the easy alteration of an assessment procedure into the workflow through manipulation of the database elements that specify the procedures. Relative information and meta-data associated with a selected procedure including the dimensions of VF that will be identified, equipment requirements, duration, and overviews and instructions for the proper administration of the procedure are included, and able to be manipulated and tailored to specific assessment environments. Additionally, this system allows for the inclusion of detailed step-by-step instructions for both the responder and patient, as well as areas for instructional videos and links to external sources. These are all included in each assessment's database specification file, and ingested by the ADVISOR assessment display parser.

This framework ingests information from the database that detail each step for the selected procedure. This specification data contains primitive values including text and numerical information, so they can be serialized/deserialized and accessed directly as objects within both the Unity engine and Android application. Then, depending on the inputs contained within these files, ADVISOR generates the appropriate procedure documentation screens and user interface (UI) elements within the Android application. This is made possible by a mapping between the UI elements created within Android's view XML specification, and fields that are present within the assessment method database table detailing the assessment. FIG. 4 depicts an example 400 of a flexible documentation framework architecture, detailing the database specifications file for each procedure, an example of the ADVISOR assessment display parser that ingests information and maps tagged content to UI elements, and an example of a resulting ADVISOR generated user interface for an embodiment of the present disclosure. The values for each of these data fields dictates whether the mapped UI element will appear (e.g., if the value is set to 0 in the database for trial duration, the trial duration configuration UI element will not appear, as 0 is a signal to the system to defer to the assessments pre-configured defaults). This consideration of mapped values occurs as each view in the Android application is generated and the data is loaded from the database, and is conducted by the developed component known as the ADVISOR assessment display parser (FIG. 4). On the other hand, in the previous example, if it was desired for the trial duration to be a configurable option for the patient, the option could bed set it to a value that would act as the default (e.g., 3 seconds), and then a slider would appear allowing this value to be changed. In addition to determining the presence of UI elements, at run-time the scripts will parse the complete data object returned by the database, and populate all the mapped UI elements appropriately including all text and image content. This allows a single and consistent information screen created within the Android application to be reused for each assessment, but have a variable number of configurable variables and user interaction techniques. Then, when an assessment is actually launched, the system will automatically iterate through each of the presented UI interaction elements and fetch their tag and value, which is used to store on the launch intent when switching applications so the data is effectively passed between the two applications.

The ADVISOR procedure administration and documentation framework, which is constantly undergoing revisions and enhancements for enhanced flexibility, results in the generation of the above outlined workflow. First, it displays various aspects of procedures on the ADVISOR application's main menu and description views, allowing for a view of the requirements and strengths of the assessment. The responder can then advance further into the instructions for this procedure by intuitively selecting assessments in a list, being shown additional detailed instructions and videos, pictures, and additional configuration options if this has been included within the procedure's specification database table, where a mapping between UI elements and data fields is utilized to hide or show various elements. This shared component utilized in various locations throughout the application, but is also supplemented by hard-coded content for various procedures, where direct manipulation of the content may not be necessary or desired due to their explicit specifications (e.g., the header fields).

Ray Casting and Collision Library: As the ADVISOR system is intended to transition traditionally kinetic and physical exams to a three dimensional environment, it was created with the ability to track patient head direction to impose constraints for both the proper and accurate administration of the procedures, as well as safety considerations. Fortunately, the Oculus API can be used within Unity to tie patient HMD movement to the cameras within the scene, being able to detect precisely the direction, angle, and rotation of the patient's head position. The implementation of this capability is necessary because many vestibular procedures require the patient to remain forward facing throughout the entire assessment, tracking objects with just eye movements. Adjustments to the forward facing position need to be recorded, and handled differently based on the specifications of each procedure. In practice, patient head movement is a fairly subjective measure, with the responder deciding the degree of change to head position which constitutes a significant enough movement for a negative result. The ADVISOR application implements complex ray casting and head position tracking based on the position of the HMD, allowing recording of the exact amount of patient head movement during respective assessment procedures.

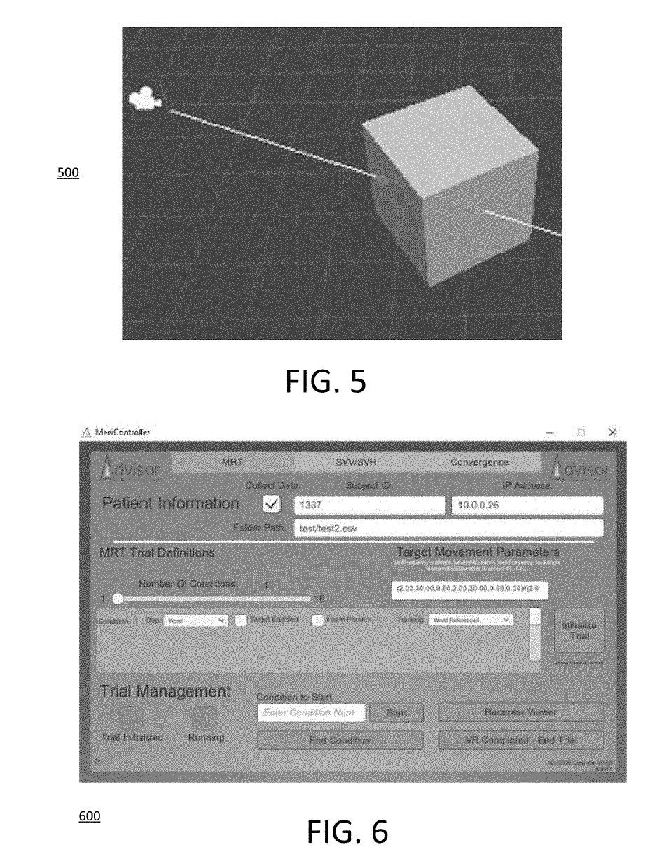

FIG. 5 depicts an example 500 of ADVISOR ray casting and collision library that allows for the tracking of patient head movement by casting rays into the virtual environment originating from the focal eye points (represented by the camera) for an embodiment of the present disclosure. Ray casting deals with the utilization of rays, which are essentially directed lines originating from the focal eye-points (i.e., the left and right eye cameras within the HMD) and tracking out into the virtual world indefinitely (FIG. 5).

Casting is a commonly used term when dealing with three-dimensional virtual environments and volumetric projection and visualizations, and involves the intersection of rays with various objects within the virtual environment. The implementation of this head tracking capability focused on the maintenance of head position within acceptable thresholds, and these thresholds are used to construct invisible “threshold objects”, which are either Unity Spheres or Panels. These invisible objects are considered at each frame (60 times per second), and ray collisions from the patient's focal eye point are detected, assuming the patient's gaze corresponds to the position of the HMD (FIG. 5). These checks are performed at every frame due to the level of precision required to accurately assess VF with ADVISOR's virtual procedures, and these checks are conducted within the Update( ) method of the controller script tied to the scene. However, this granularity presents a significant development challenge, requiring the implementation to be efficient. The complexities of utilizing a traditional ray casting approach in a three dimensional space requires an immense amount of resources and memory storage space, and the basic prior utilizations of such technologies within Unity failed to meet the efficiency requirements. Although modern advances in graphical processing hardware has made these traditional algorithms more feasible, they are still computationally expensive, relying heavily on and quickly consuming all available video RAM. While Unity provides a ray casting implementation with its software, exclusively utilizing this library would prove inefficient and fail to provide various features necessary for clinical application, including the ability to easily access which object was intersected first (if more than one), the ability to halt ray casting once a desired object has been intersected, and other efficiency considerations. Therefore, Unity's ray casting implementation was augmented to develop the ADVISOR ray casting and collision library for these detections.

First, this library was implemented with efficiency at the forefront of development priorities, as to not create any visual lag or staggering in the virtual implementation of the procedures. The implementation ensures rays and collisions are only calculated when they are required, and not considered during any instruction screens or sections within the procedure where head tracking is not important. Therefore, each of the HMD enabled procedures contains a Boolean flag to specify if ray casting should be checked during the current frame. This flag is only enabled when ray casting should be utilized, initially being set to false and resetting to false at any period of pause or when instructions are being presented to the patient. Before conducting any sort of ray tracing or collision detection, the positional sensors on the HMD are utilized, determining if any movement has been detected from the last frame. This is an API call within the Oculus software, which allowing acquisition of the current head position (camera position) and rotation of the HMD, store this information for comparison later in a variable within ADVISOR, and compare it to the last known position. If movement has been detected (e.g., rotation in the X-, Y-, or Z-axis), the library then continues to determine and “shoot” a ray indefinitely into the virtual world utilizing Unity's Ray implementation. This ray is started by calling the Ray functions on the GameObject that represents the main camera; for the described implementation only a single camera need be considered, the one representing the left eye, since both are always facing the same direction. The ADVISOR ray casting and collision library then seeks to determine if the ray has intersected with any object within the virtual environment. Again, with efficiency in mind, the implementation of the invisible threshold objects places them in front of any other objects within the world, allowing the software to quickly determine if a collision was present due to the short distance of the required ray calculation. Additionally, once a collision with an object is detected, the ADVISOR system is immediately alerted, and the ray calculations are ceased for the sake of efficiency with a return statement and by flipping the Boolean for Ray calculation, halting the ray considerations for the current frame.

The collision data is then used to trigger a change within the assessment (e.g., informing the user with visual cues if they begin moving towards the threshold, pausing the test if the threshold is reached, informing the ADVISOR system if necessary collision has occurred, or encouraging patients to remain within acceptable bounds—Detailed in the HMD test specifications below). Utilizing the ADVISOR ray casting and collision library the system is able to monitor the movement of the patient's head, ensuring the test is administered correctly. If patient movement falls outside acceptable bounds (e.g., they are rotating too slowly, or not rotating their head enough), testing is halted based on the specifications of the procedure, and does not continue until movement has been restored to within acceptable thresholds.

Yield Return for Object Hiding/Displaying: Several of the procedure implementations require the toggling of the display and occlusion of visual or audio stimuli at specific timeframes. For example, during the Subjective Visual Vertical examination, the ADVISOR requirements call for a “rest” period of four seconds between subsequent trials, giving the patient a break before performing additional assessment. Pausing to this degree has been accomplished in the past, but has typically been handled on the main application thread, which would essentially halt the application for the duration of the pause. This is because all code needs to be executed to completion before returning or continuing. When implemented in this manner, particularly within the head mounted display (HMD) based procedures, this caused undesired effects and visual artifacts generated as a result of pausing the main UI thread, and caused disorientation because the patient could move their head within the HMD, but the scene would remain frozen. Therefore, Unity3D's co-routine capabilities are utilized, which allows tasks to be created and run in the background. This allows ADVISOR to continue its UI thread (preventing disorientation), but still allowing the timer to occlude/display objects as necessary. Co-routines are created for this application, using Unity's Yield Return capabilities combined with its WaitForSeconds functions. The implementation has the background routine wait for the specified number of seconds, and once reached, flips a Boolean flag to indicate the timer has been reached and the status of the stimuli should be toggled. This Boolean flag is checked at every frame within Unity's Update( ) method, altering the display appropriately from the main UI thread.

VR Assessment Personalization: Each of the ADVISOR HMD Patient Application procedures is followed by personalized instructions that include the patient's ID and responder's name, and inform the patient to remove the HMD and return the device to the responder. ADVISOR automatically detects this removal by utilizing an API call to the Oculus API to determine if both the current procedure is complete, and if the HMD is not currently on the patient's face. If both these variables are correct, the shared component Application Switcher is utilized to return the device to the paused Responder Application, after saving the results object persisting the data to the device. On the resuming of the Responder Application, the results file is automatically parsed, and the new procedure data is obtained. If the data exists (meaning the HMD procedures were completed without error), ADVISOR will automatically store this data to the secure server for the specified procedure.

Remote Control and Configuration: In addition to the standard ADVISOR framework for viewing, selecting, and configuring assessments on the Android application, then launching the VR application for the trials, and then viewing the results back on the Android application, the ability for remote control of the entire system was included for an implemented embodiment. FIG. 6 depicts an example 600 of a control interface that can be used to manipulate a target's trajectory and different trials within an assessment for an embodiment of the present disclosure. This feature enables multiple-device usage and control through a single controller interface, like the exemplar interface shown in FIG. 6, designed to enable experimental trials to be run on the ADVISOR suite.

The option for remote control can be enabled through checkbox in the settings configuration on the main Android application, which when enabled, begins a UDP client background service that will listen for incoming messages on a specific port. External applications or clients can then send UDP messages to that singular device, or use the broadcast IP address (.255) to broadcast to multiple devices. In order for the messages to be received and to be acted on by the ADVISOR system, it needs to be structured to a specific format, with instruction and configuration variables combined into a single string, containing all the information that would be sent to the VR application if a standard launch was conducted. Upon receiving a valid UDP message, the ADVISOR Android application will launch the VR application to the specified assessment, but place the assessment into a remote control mode, having its timings and start/stop/data collect all dictated by the background running Android application. The Android application, now running in the background, will continue to communicate with the VR application as additional UDP messages are received through the use of Broadcast Intents, which the VR application can receive and respond to via an Android-based Unity plugin. Again, this is structured this way to promote full control for use in controlled experiments, where timings may need to be synchronized across multiple systems or pieces of hardware. Start, stop, and end messages are common among the assessments, allowing the application to successfully run through a VR assessment and then return as normal to the Android device. Data storage from the trial occurs when an end message is received, so data is still saved despite this new control interface.

Passthrough Camera Implementation: During the creation of the remote control interface to support experiment running with the ADVISOR system, it became apparent that patients may be forced to wear the HMDs for extended periods of time, which may cause disorientation or confusion. Therefore, the ability to have a passthrough camera on any of the VR assessments was included for an implemented embodiment; the passthrough camera can be triggered through the flipping of a simple Boolean variable. If this Boolean is enabled, the device's camera is activated, and its feed is displayed directly onto a flat texture attached to the patient's camera within the Unity3D environment. The effect is that of an augmented reality display—they are able to see the real world around them despite wearing the VR goggles. In current practice this is typically used as a standby screen between different trials, but has also been utilized for various experimental trials where virtual objects (e.g., a target moving around the screen) are overlaid over this real world representation.

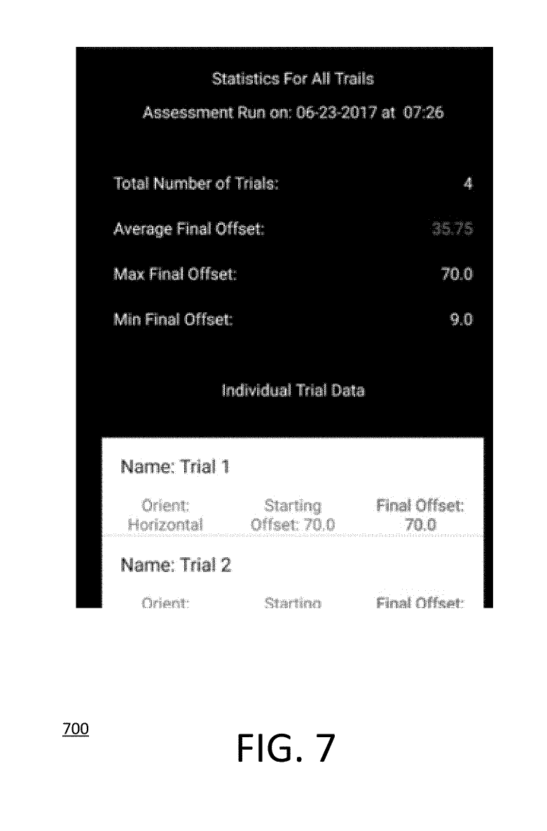

Review Performance: The ability to review the performance of a patient on each assessment or across multiple assessments is a crucial part of the ADVISOR system. Each assessment will have its own results screen, as the dimensions that should be represented within this screen vary across each vestibular assessment. However, these views will all be similar, following the same UI format for consistency. They will contain the procedure name at the top, and be followed by the data specific to the selected assessment (e.g., DVAT needs to display distance from the user, while the SVV procedure displays rotational offsets). This allows an extremely flexible and reusable framework for displaying information. An example 700 of some initial results from a single run of the Subjective Visual Vertical assessment can be seen in FIG. 7.

### Body Positioning and Movement

ADVISOR records motion data of the arms, legs, and torso of patients undergoing neurological function tests. Recording motion data enables real-time or post hoc analysis of movement and the development of quantifiable measures of neurological function derived from exams that assess balance, gait, or voluntary/involuntary movement of the body or extremities.

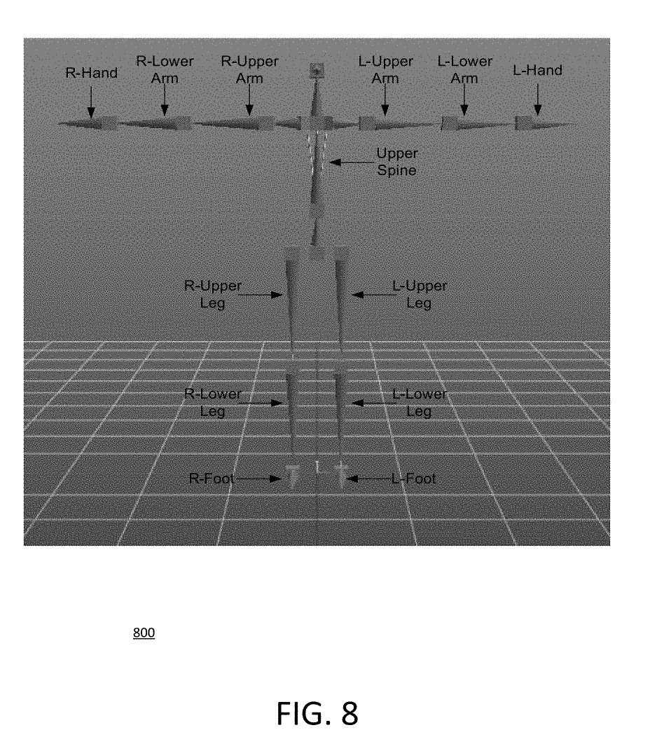

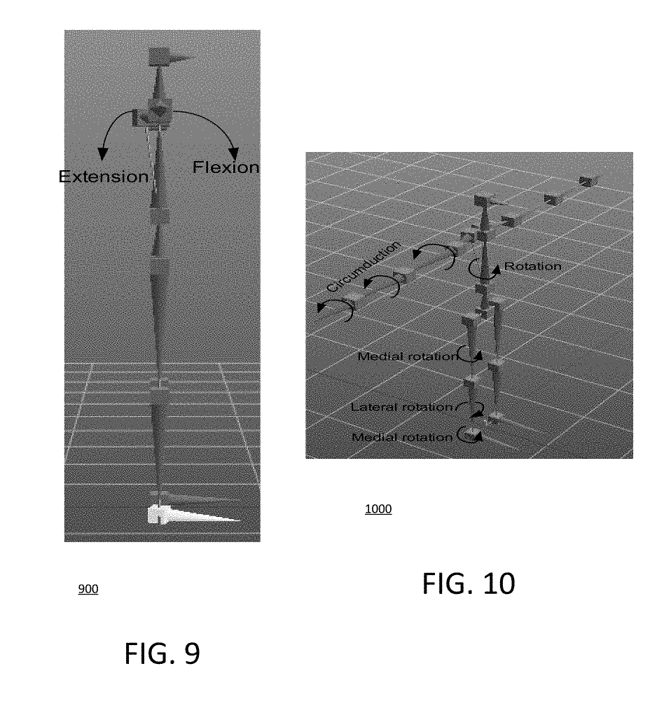

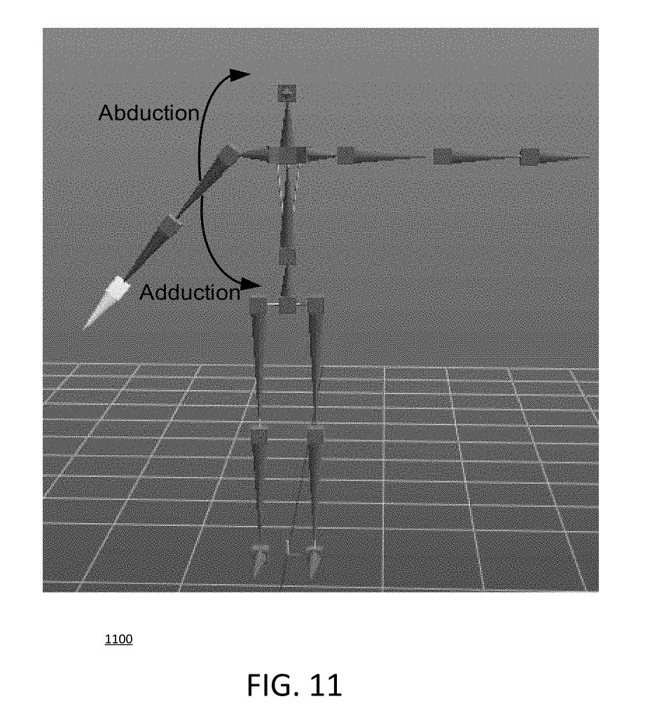

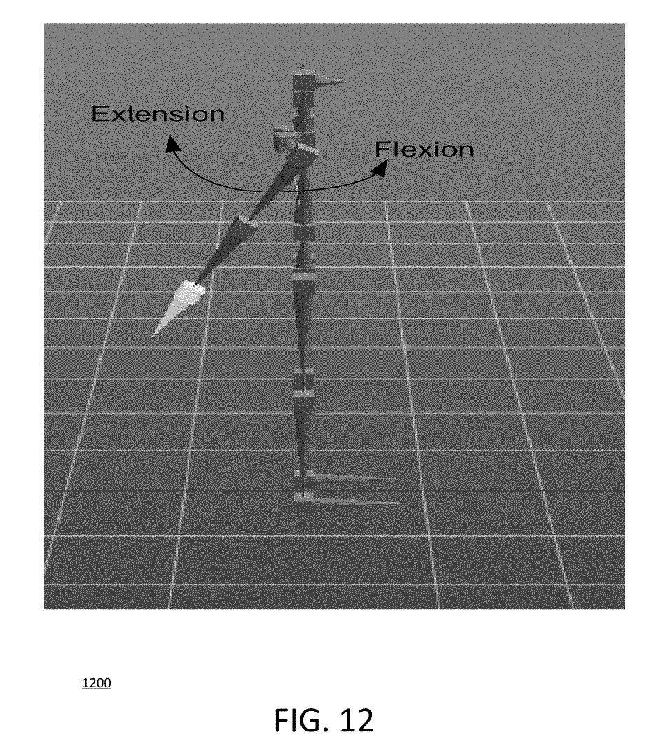

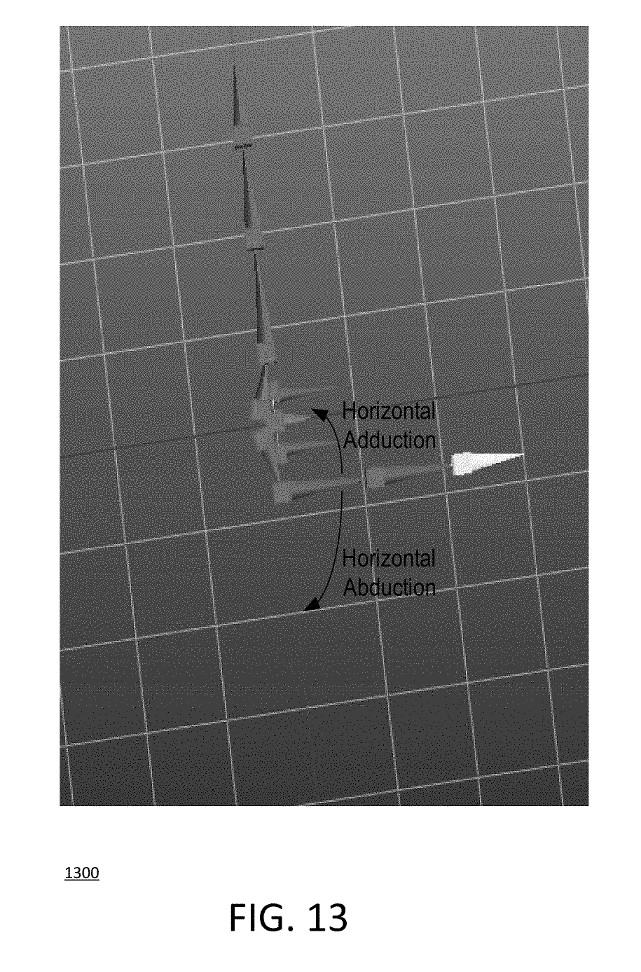

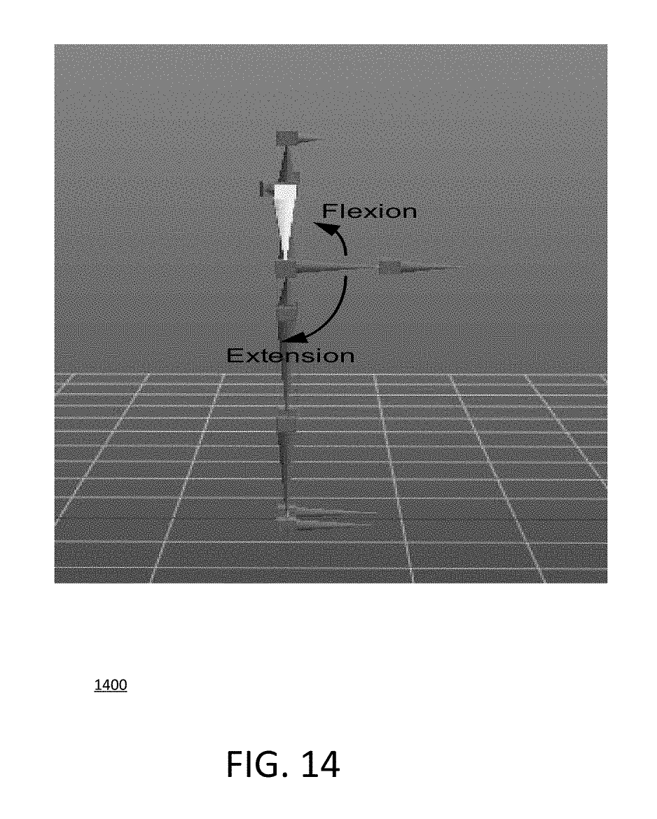

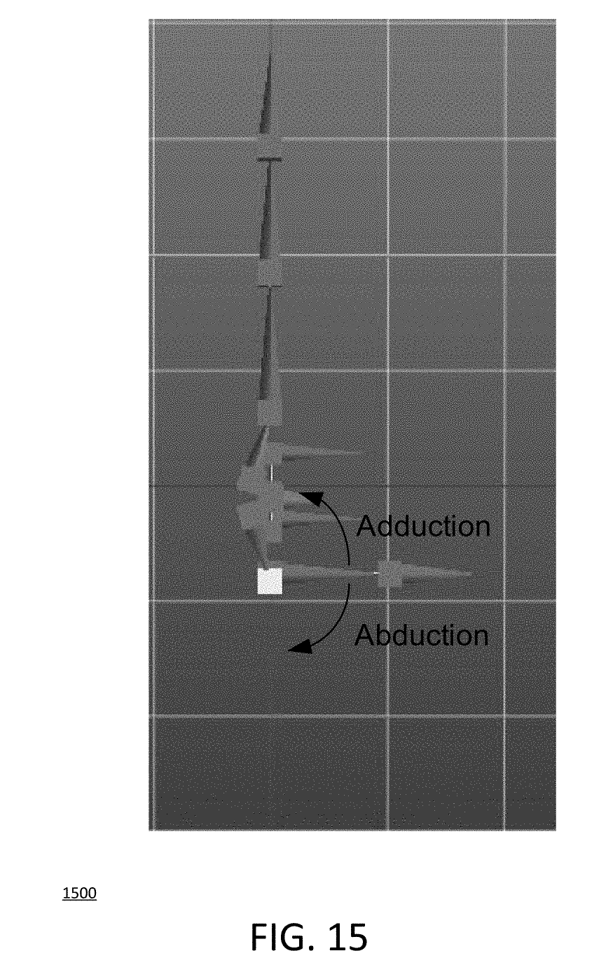

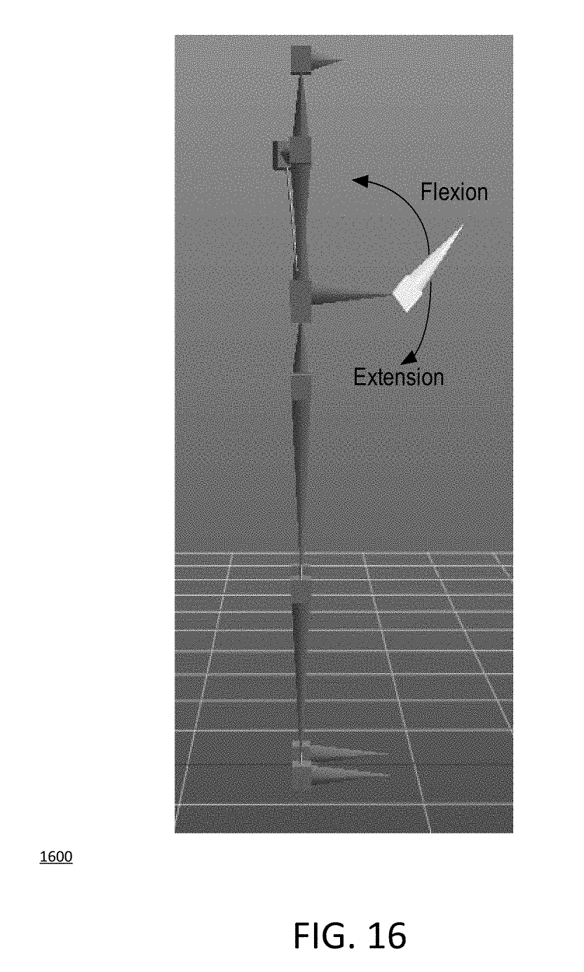

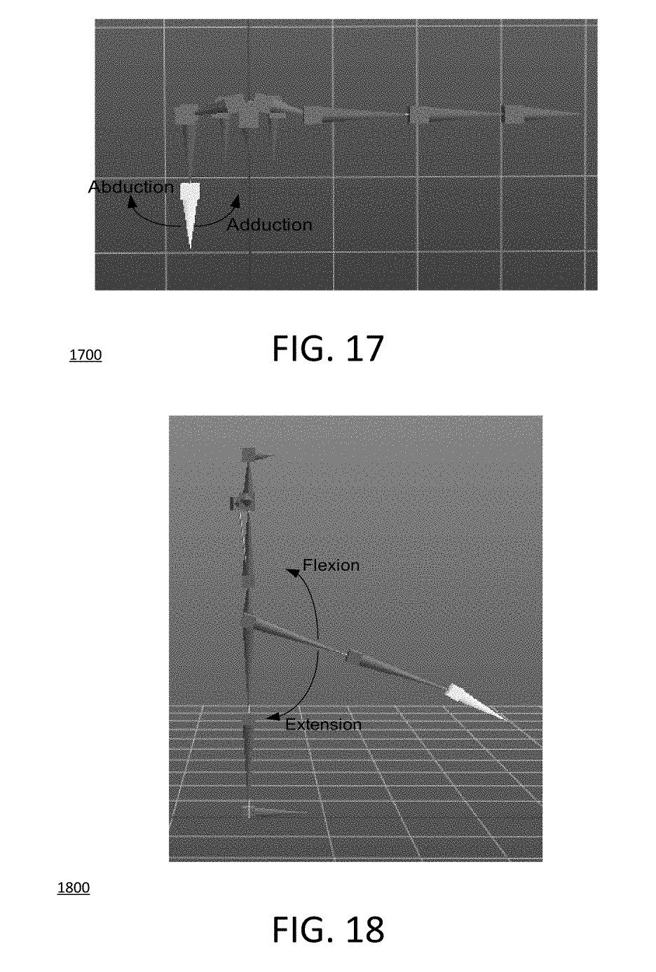

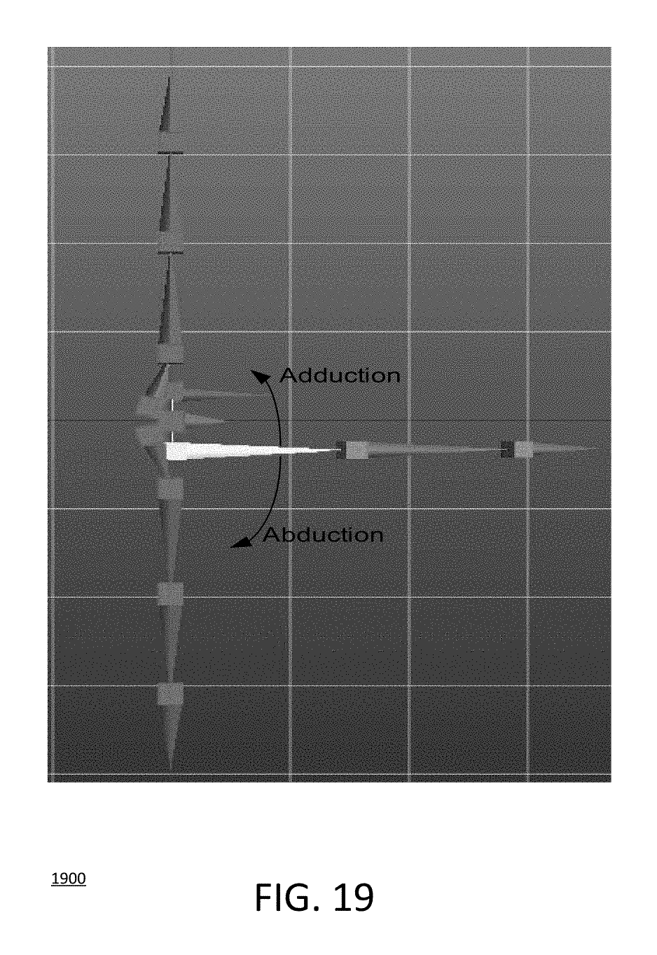

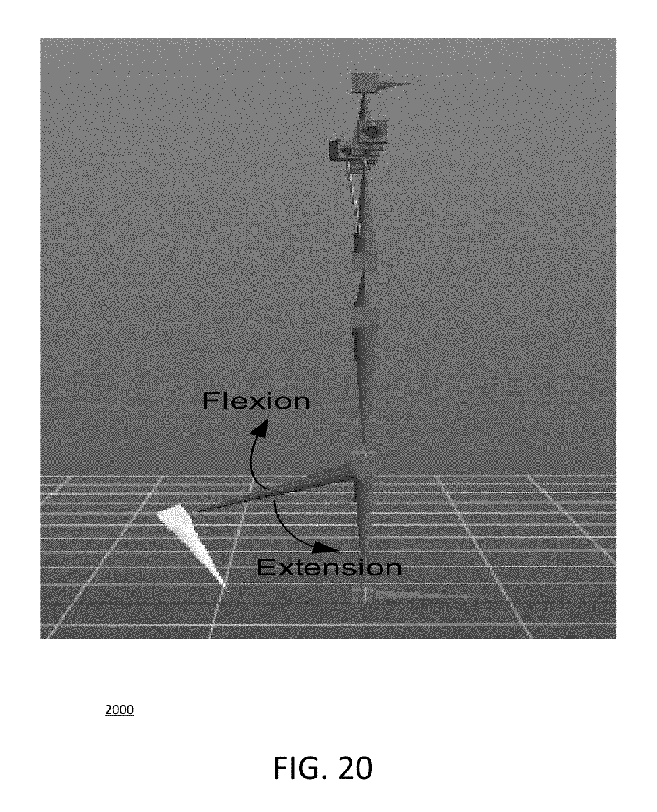

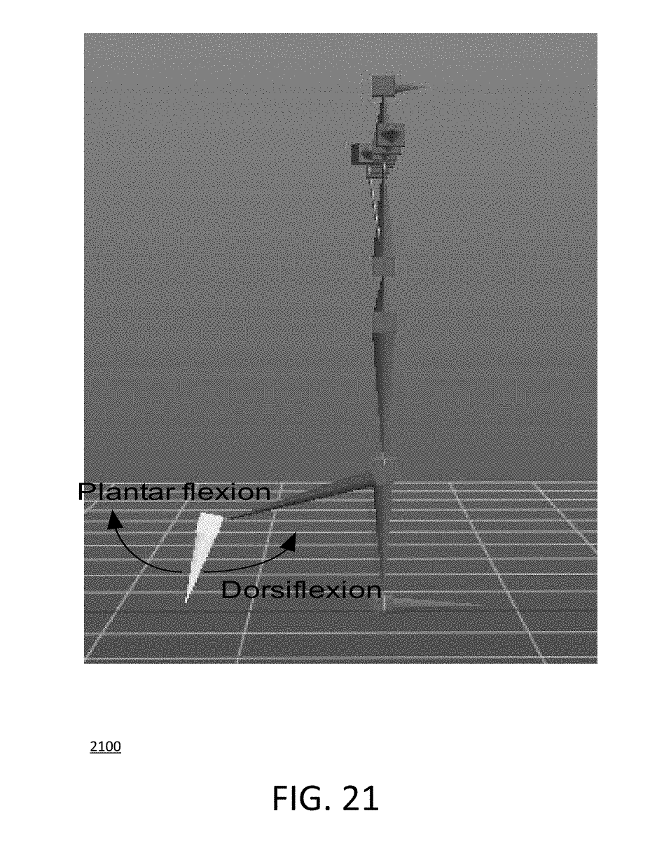

ADVISOR does not rely on specific hardware technology to record motion capture data. However, any motion capture hardware used with ADVISOR preferably meets the requirements outlined in the paragraphs below. Motion capture hardware used with ADVISOR must provide quaternion output describing the rotation and position of individual components of a wire-frame skeleton. The hardware must have an API compatible with the Unity3D Gaming Engine version 4 or higher. FIG. 8 depicts an example 800 of wireframe components used for embodiments of the present disclosure. The motion capture hardware used with ADVISOR must be capable of providing quaternion information for each element of the wire-frame skeleton called out in FIG. 8. FIGS. 10 through 21 show the movements for each skeleton component that the motion capture hardware needs to be capable of detecting. Table 1 summarizes this information:

TABLE 1
Summary of motion capture requirements for ADVISOR hardware

Wire Frame Component

| | |
|---|---|
| Upper arm | Abduction and adduction |
| | Horizontal abduction and horizontal adduction |
| | Extension and flexion |
| | Circumduction |
| Lower arm | Abduction and adduction |
| | Extension and flexion |
| | Circumduction |
| Hand | Abduction and adduction |
| | Extension and flexion |
| | Circumduction |
| Upper spine | Rotation |
| | Extension and flexion |
| Upper leg | Abduction and adduction |
| | Extension and flexion |
| | Lateral rotation |
| | Medial rotation |
| Lower leg | Extension and flexion |
| Foot | Planter flexion and Dorsiflexion |

ADVISOR is unique in its application of motion capture sensing to neurological function testing to provide a quantifiable means of assessing patient condition. Many neurological function tests currently rely on the subjective observations of the test administrator to determine performance. Subjective observation provides no way to identify small changes in a patient performance over time. Subjective observation of performance also provides no way for two different people administering the same test to reconcile their assessment of the patient's performance. One person might think a patient's performance is within normal bounds, while another does not.

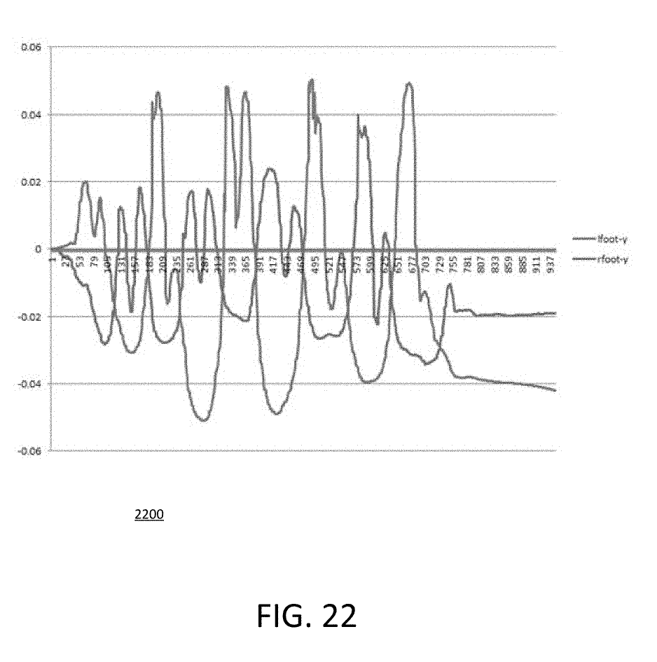

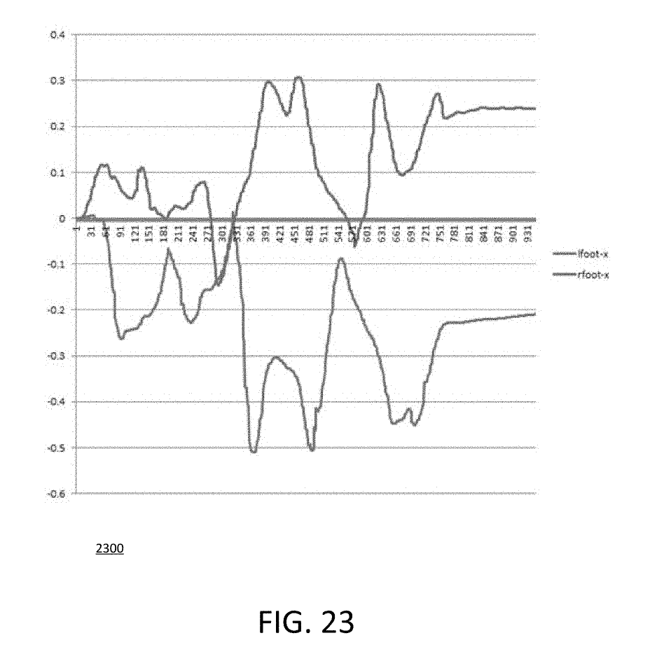

ADVISOR can record motion capture data from the wire-frame components listed in FIG. 8. The recorded data can then be examined to extract relevant information. FIGS. 22 and 23 show data that ADVISOR recorded about foot position, captured during a Fukuda Stepping Test. The Fukuda Stepping test is designed to assess neurological function. It requires a patient to close their eyes and walk in a straight line. A patient with neurological issues will drift to one side or the other. ADVISOR uses the data below to determine how far a patient drifts right or left during the test. This data provides a quantifiable measure of a patient's performance, allowing multiple test administrators to compare an individual's test results across multiple test instances performed over a period of time and determine if a patient's neurological condition is improving or not.

ADVISOR records data for movement of all extremities as well as the chest and torso. The aggregated data set provides enough information to apply a quantifiable measure of patient performance for neurological tests that assesses balance, gait, or voluntary/involuntary movement of the body or extremities.

### Exemplary Embodiments and Additional Features

Exemplary embodiments of the present disclosure can provide support for the tethering of Wii Balance Board via Bluetooth to an Android Platform to measure and record center of pressure and center of gravity of an individual. Exemplary embodiments of the present disclosure can provide a Wii Balance Board synchronization and data processing library for Android connection, written on the Bluetooth HID wireless protocol, along with inclusion of data recording capabilities and live visualizations of performance. Exemplary embodiments of the present disclosure can provide a synchronization and data processing library for Leap Motion's new Location and Spatial Mapping sensor to enable spatial mapping of the real world environment to the VR environment, including Utilization of this sensor for realistic VR movement around environments. Exemplary embodiments of the present disclosure can provide an iOS based version of the ADVISOR application suite including porting all necessary plugins and data collection libraries to the iOS platform. Exemplary embodiments of the present disclosure can provide the ability to aggregate multiple assessment results together to provide a more robust diagnosis of vestibular health. Exemplary embodiments of the present disclosure can provide a Windows Augmented and Mixed Reality implementation of the application suite, allowing ADVISOR assessments to be conducted on augmented and mixed reality systems such as the Microsoft HoloLens, and the Acer and Lenovo Mixed Reality Headsets. Exemplary embodiments of the present disclosure can provide the ability to track saccadic eye movements inside a head-mounted display with a custom solution providing upwards of 10 kHz sampling rate. Exemplary embodiments of the present disclosure can provide an integrated Camera-based eye tracking solution with sample rates and image-based collection of up to 500 Hz. Exemplary embodiments of the present disclosure can provide support for EMG data collection over Wi-Fi or Bluetooth, including EMG sensor synchronization and data collection library for use in the ADVISOR suite. Exemplary embodiments of the present disclosure can provide the ability to detect VEMPs from ocular or cervical muscles. Exemplary embodiments of the present disclosure can provide a Synchronization and control library for Bluetooth based, non-location specific, haptic pulse generator that can be applied on any part of the body and triggered by the ADVISOR suite. Intuitive visualizations of vestibular assessment results to provide at-a-glance summaries of vestibular health and recommendations for future care. Further, exemplary embodiments of the present disclosure can provide Integration of VR motion controllers to provide intuitive user interactions and control of assessments.

Further exemplary embodiments are described in the following numbered clauses; where not mutually exclusive, the subject matter of any of clauses 2-33 can be combined:

Clause 1: a system A software framework for developing and deploying stimulus-response (SR) based health assessment methods, the framework including:
A flexible and customizable procedure administration and documentation user interface architecture developed and deployed to aid in the identification, administration, configuration, and instruction of a suite of health assessment procedures;
A Unity3D-based virtual reality environment configured so as to enable the accurate audiovisual presentation of stimulus for different health assessments to trigger target user responses;
Software harness for integration of hardware input peripherals (e.g., positional sensors) to enable user response acquisition;
A database storage and retrieval backend configured to logically store individual trial assessment.

Clause 2: The system of claim 1, whereby an online PostgreSQL database is used for storage of procedure information.

Clause 3: The system of claim 2, whereby a configuration interface is available to enable intuitive changes, additions, or deletions to the content of the smartphone application.

Clause 4: The system of claim 2, further including a standardized mapping between the database fields and the XML, code that comprises the interface, affording the ability to show or hide content by changing fields within the database.

Clause 5: The system of claim 1, further including a robust local smartphone data storage and scanning system for local persistence of data to enable redundant data storage.

Clause 6: The system of claim 1, further including an optional client application for remote control and configuration of health assessments on a smartphone or other mobile device.

Clause 7: The system of claim 6, further including User Datagram Protocol (UDP) based messaging for control, allowing any properly configured device to utilize the ADVISOR system remotely.

Clause 8: The system of claim 7, further including low-latency message transmission over any public or private network.

Clause 9: The system of claim 1, further including the ability for sensor data is captured at rates beyond the standard capabilities of Unity3D through the use of Java-based plugins which operate on the native operating system and are not subject to the limitations of Unity (e.g., 60 Hz capture rate on external sensors).

Clause 10: The system of claim 1, further including Java-based plugins allowing for access to native operations on mobile devices such as refreshing of the file system or manipulation of the application stack.

Clause 11: The system of claim 1, further including a user interface to facilitate intuitive health assessment method selection, understanding, execution, and results analysis.

Clause 12: The system of claim 11, further including common XML formatting, allowing for easy addition and alterations to each user interface.

Clause 13: The system of claim 11, further including XML interface elements mapped to database fields for population and to determine display contents.

Clause 14: The system of claim 11, further including information flow protocols to transmit database content to an XML parser, which decides its presentation based on a coded value, allowing future alterations to the database to visually change the user interface without manipulations to the codebase.

Clause 15: The system of claim 1, wherein rule-based analytics can be incorporated to integrate the results of multiple assessment trial and/or completed assessment results.

Clause 16: The system of claim 15, further including PostgreSQL data storage to enable data aggregation and speedy retrieval of numerous records using SQL queries with near-zero latency.

Clause 17: The system of claim 1, whereby the stimulus presentation solution can be deployed to any smartphone or other computing platform supported by the Unity3D game engine.

Clause 18: The system of claim 13, further including augmentations to Unity3D's standard Raycasting library to afford more efficient collision detection and higher display frame rates while still allowing for complex gaze and movement detection.

Clause 19: The system of claim 13, further including utilization of Unity's Input system for management of controller input to capture explicit patient responses.

Clause 20: The system of claim 1, further including a user account creation and user login authentication capabilities to restrict user access privileges.

Clause 21: The system of claim 16, further including an online NodeJS server, implementing common libraries such as, Express for routing, and Sequelize for database and object model support.

Clause 22: The system of claim 20, further including PassportJS code to create a robust authentication system using Password-Based Key Derivation Function 2 (PBKDF2) cryptography.

Clause 23: The system of claim 16, further including authentication standards ensure proper credentials at every operation (i.e., not just during initial login) on the server.

Clause 24: The system of claim 1, further including configuration settings to specify user profile details relevant to health assessments (e.g., demographics, anthropometrics).

Clause 25: The system of claim 24, further including online storage of profile data that can be accessed on demand by smartphone application services (e.g., assessments that require demographic data for interpretation).

Clause 26: The system of claim 1, further including the ability to collect data from any Bluetooth supported third-party sensor.

Clause 27: The system of claim 26, further including serial Bluetooth connections to ensure adaptability with any commercially available Bluetooth-capable sensor.

Clause 28: The system of claim 1, further including the ability to associate IMU data to a skeletal model of an individual's body segments on the smartphone.

Clause 29: The system of claim 28, further including the capability to deploy IMUs as required to only track specific segments of an individual's body motions.

Clause 30: The system of claim 28, further including automated algorithms to calculate joint angles, accelerations, limb positions in space, and orientation.

Clause 31: The system of claim 28, further including the ability to capture raw quaternion information on each skeletal segment position.

Clause 32: The system of claim 28, further including the ability to record and transmit to the online database all recorded IMU data associated with body segment position and movements.

Clause 33: The system of claim 28, further including the ability to control a virtual avatar within Unity3D when appropriate virtual model rigging is designed as part of the virtual skeletal model.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

For example, the embodiments of the described systems/methods can be utilized for rehabilitation purposes by providing users with a series of ocular and balance-related exercises driven by VR stimulus, with connected sensors then used to monitor rehabilitation progress and compliance. A system according to the present disclosure can also be used for other types of user assessments such as visual acuity assessments (using visual stimulus within the VR headset to elicit user responses that can be used to determine visual acuity and field of view) or hearing assessments (using the already incorporated audiology features to assess user hearing thresholds). The described systems/methods can also be readily used for exercise purposes, to provide motivational content to promote exercise compliance. For example, utilizing immersive VR environments to give a user the sense that they are working out on a beach, and using connected sensors to confirm users are executing different yoga positions correctly. Embodiments of the described systems/methods can also be used for strictly cognitive assessments, by incorporating already validated cognitive assessments, such as those of the NIH Toolbox, to provide a portable platform for cognitive capabilities assessment. Further, embodiments of the described systems/methods can also be used as a portable training platform, using visual and auditory stimulus to instruct users on how to execute different physical tasks, and then using connected sensors to monitor performance and provide feedback to promote compliance.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element proceeded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

What is claimed is:

1. A system for deploying stimulus-response (SR) based health assessment methods for assessing the health of a subject, the system comprising:
a flexible and customizable procedure administration and documentation user interface architecture operative to present a plurality of health assessment procedures to an evaluator;
a virtual reality environment configured to enable the accurate audiovisual presentation of stimulus for different health assessments to trigger responses from a subject;
a plurality of positional sensors operative to acquire data of the subject's stimulus-responses;
a computer-readable non-transitory storage medium, including computer-readable instructions; and
a processor connected to the memory and operative to evaluate the subject's stimulus-responses, wherein the processor, in response to reading the computer-readable instructions, is operative to: evaluate the subject's stimulus-responses, and present the evaluation to the evaluator.

2. The system of claim 1, further comprising a database storage and retrieval server configured to logically store individual trial assessment in a database.

3. The system of claim 2, wherein the database comprises an online PostgreSQL database is used for storage of procedure information.

4. The system of claim 2, wherein a configuration interface is available to enable intuitive changes, additions, or deletions to the content of the smartphone application.

5. The system of claim 2, further including a standardized mapping between the database fields and the XML code that comprises the interface, affording the ability to show or hide content by changing fields within the database.

6. The system of claim 1, further including a robust local smartphone data storage and scanning system for local persistence of data to enable redundant data storage.

7. The system of claim 1, further including an optional client application for remote control and configuration of health assessments on a smartphone or other mobile device.

8. The system of claim 6, further including User Datagram Protocol (UDP) based messaging for control, allowing any properly configured device to utilize the ADVISOR system remotely.

9. The system of claim 7, further including low-latency message transmission over any public or private network.

10. The system of claim 1, further including the ability for sensor data is captured at rates beyond the standard capabilities of Unity3D through the use of Java-based plugins which operate on the native operating system and are not subject to the limitations of Unity (e.g., 60 Hz capture rate on external sensors).

11. The system of claim 1, further including Java-based plugins allowing for access to native operations on mobile devices such as refreshing of the file system or manipulation of the application stack.

12. The system of claim 1, further including a user interface to facilitate intuitive health assessment method selection, understanding, execution, and results analysis.

13. The system of claim 11, further including common XML formatting, allowing for easy addition and alterations to each user interface.

14. The system of claim 11, further including XML interface elements mapped to database fields for population and to determine display contents.

15. The system of claim 11, further including information flow protocols to transmit database content to an XML parser, which decides its presentation based on a coded value, allowing future alterations to the database to visually change the user interface without manipulations to the codebase.

16. The system of claim 1, wherein rule-based analytics can be incorporated to integrate the results of multiple assessment trial and/or completed assessment results.

17. The system of claim 15, further including PostgreSQL data storage to enable data aggregation and speedy retrieval of numerous records using SQL queries with near-zero latency.

18. The system of claim 1, whereby the stimulus presentation solution can be deployed to any smartphone or other computing platform supported by the Unity3D game engine.

19. The system of claim 13, further including augmentations to Unity3D's standard Raycasting library to afford more efficient collision detection and higher display frame rates while still allowing for complex gaze and movement detection.
